(19) United States
(12) Patent Application Publication
(10) Pub. No.: US 2024/0398816 A1
(43) Pub. Date: Dec. 5, 2024

(54) Gel Treatment for Loss of Taste and Smell

(71) Applicant: Cyrano Therapeutics, Inc., Delray Beach, FL (US)

(72) Inventors: Richard GEOFFRION, Delray Beach, FL (US); Ronald KUPPERSMITH, College Station, TX (US)

(21) Appl. No.: 18/742,344

(22) Filed: Jun. 13, 2024

Related U.S. Application Data
(63) Continuation of application No. PCT/US2022/081646, filed on Dec. 15, 2022.
(60) Provisional application No. 63/424,584, filed on Nov. 11, 2022.
(60) Provisional application No. 63/292,028, filed on Dec. 21, 2021.

(51) Int. Cl. A61K 31/522; A61K 9/00; A61K 9/06; A61K 47/32; A61K 47/38; A61P 27/00

(52) U.S. Cl. CPC A61K 31/522; A61K 9/0043; A61K 9/06; A61K 47/32; A61K 47/38; A61P 27/00

(57) ABSTRACT

Disclosed herein are methods of treating chemosensory dysfunction in a subject, comprising administering to the subject a gel comprising a phosphodiesterase inhibitor. Also disclosed herein are methods of treating chemosensory dysfunction by administering a phosphodiesterase inhibitor to a subject in need thereof. Also disclosed herein are kits comprising gels.

### CROSS-REFERENCE

This application is a continuation of PCT/US2022/081646, filed Dec. 15, 2022, which claims the benefit of U.S. Provisional Application No. 63/292,028, filed Dec. 21, 2021, and of U.S. Provisional Application No. 63/424,584, filed Nov. 11, 2022, the disclosures of which are incorporated herein by reference in their entirety.

### SUMMARY

This disclosure relates to methods and compositions useful for treating or preventing chemosensory dysfunction (e.g., anosmia or dysgeusia).

For example, in one aspect this disclosure provides a method of treating or preventing chemosensory dysfunction in a subject by delivering a pharmaceutical gel composition to a nasal cavity or a sinus cavity of the subject, wherein the gel pharmaceutical composition includes a PDE inhibitor or the salt thereof and is configured to release the PDE inhibitor or the salt thereof for at least 24 hours, and further includes releasing the PDE inhibitor or the salt thereof in the nasal cavity or the sinus cavity of the subject, thereby treating or preventing chemosensory dysfunction in the subject.

In some embodiments, the PDE inhibitor or salt thereof can include at least one of theophylline or a salt thereof, roflumilast or a salt thereof, or cilostazol or a salt thereof. In some embodiments, the PDE inhibitor or salt thereof can include theophylline or the salt thereof, and the pharmaceutical gel composition can be configured to release the theophylline or the salt thereof at a dosage of about 20 μg to about 2000 μg per day. For example, in some embodiments, the PDE inhibitor or salt thereof can include theophylline or the salt thereof, and the gel composition can be configured to release theophylline or the salt thereof at a dosage of about 5 μg to about 400 μg per day for at least about 4 days. In other embodiments, the PDE inhibitor or salt thereof can include theophylline or the salt thereof, and the pharmaceutical gel composition can be configured to release theophylline or the salt thereof at a dosage of about 20 μg to about 500 μg per day for at least about 4 days.

In some embodiments, the PDE inhibitor or salt thereof can be present in an amount from about 0.01% to about 5% weight to weight (wt/wt) of the total gel composition. In some embodiments, the pharmaceutical gel composition can include an excipient, for example, polyethylene glycol (PEG) or water. In some embodiments, the pharmaceutical gel composition can include a poloxamer, or sucrose acetate isobutyrate (SAIB), or the poloxamer and the SAIB, and wherein the poloxamer or the SAID or both can each be independently present in an amount from about 1 to about 10% weight to weight (wt/wt) of the total gel composition.

In some embodiments, the pharmaceutical gel composition can comprise a penetration enhancer. For example, the penetration enhancer can comprise a fatty acid, an alcohol, a surfactant, a solvent or any combination thereof. In some embodiments, the pharmaceutical gel composition can comprise the penetration enhancer in an amount from about 0.10% to about 20% weight to weight (wt/wt) of the total composition.

In some embodiments, the pharmaceutical can be delivered to the sinus cavity, for example, an ethmoid sinus cavity, a maxillary sinus cavity, a frontal sinus cavity, or a sphenoid sinus cavity. In some embodiments, the pharmaceutical can be delivered to the upper nasal cavity, the olfactory cleft, the olfactory epithelium or any combination thereof. In some embodiments, the chemosensory dysfunction can be ageusia, hypogeusia, dysgeusia, parosmia, phantosmia, anosmia, hyposmia, dysosmia, or any combination thereof. In some embodiments, the chemosensory dysfunction can be a condition that arises after or during a viral infection, for example, such as a coronavirus infection or an influenza infection. In some embodiments, the subject can be diagnosed with a chemosensory dysfunction. Accordingly, in some embodiments, the subject that is treated by the method can be a subject in need of the pharmaceutical gel composition.

According to certain embodiments, the pharmaceutical gel composition can be delivered to both nostrils of the subject. In other embodiments, the gel composition can be delivered to a single nostril.

In another aspect, this disclosure provides a pharmaceutical gel composition comprising a PDE inhibitor or a salt thereof, wherein the gel composition is configured to release the PDE inhibitor or the salt thereof for at least 24 hours in a subject that is in need thereof. In another aspect, this disclosure provides a comprising the pharmaceutical gel composition and a container.

In a different aspect, this disclosure provides a method of treating or preventing chemosensory dysfunction in a subject, the method comprising: delivering a gel composition to a nasal cavity or a sinus cavity to treat or prevent chemosensory dysfunction in the subject, wherein the gel composition comprises: (a) a poloxamer; (b) a sucrose acetate isobutyrate (SAIB); or (c) both (a) and (b), and wherein the gel comprises a PDE inhibitor or a salt thereof and wherein the gel is configured to release the PDE inhibitor or the salt thereof for over 24 hours. In certain embodiments, the method can comprise a poloxamer. In other embodiments, the method can comprise a sucrose acetate isobutyrate (SAIB). In different embodiments, the method can comprise a poloxamer and SAIB.

In some embodiments, the PDE inhibitor or a salt thereof can comprise theophylline or a salt thereof. In some embodiments, the PDE inhibitor or the salt thereof can comprise cilostazol or a salt thereof. In some embodiments, the PDE inhibitor or the salt thereof can comprise roflumilast or a salt thereof.

In certain embodiments, a gel composition can comprise a poloxamer, a SAIB, or both, in an amount from about 1 to about 10% weight to weight (wt/wt) of the total gel composition. In some embodiments, the gel composition can comprise a PDE inhibitor or salt thereof in an amount from about 0.01% to about 5% weight to weight (wt/wt) of the total gel composition. In some embodiments, a gel composition can further comprise a penetration enhancer. In some embodiments, a penetration enhancer can include a fatty acid, an alcohol, a surfactant, a solvent or any combination thereof. In some embodiments, a gel composition can comprise a penetration enhancer in an amount from about 1% to about 20% weight to weight (wt/wt) of the total composition. In some embodiments, a sinus cavity can include an ethmoid sinus cavity, a maxillary sinus cavity, a frontal sinus cavity, or a sphenoid sinus cavity. In some embodiments, the gel composition can be delivered to the nasal cavity, for example, the upper nasal cavity, the olfactory cleft, the olfactory epithelium or any combination thereof.

In some embodiments, a gel composition can be configured to release a PDE inhibitor or the salt thereof over a period of time from about 1 day to about 14 days. For example, in some embodiments, a gel composition can be configured to release about 500 μg/ml (milliliter) of the PDE inhibitor or salt thereof per day. In some embodiments, a gel composition can be in unit dose form.

In some embodiments, a gel composition can be administered as needed, for example, for about a week, two weeks, three weeks, a month, two months, three months, four months, five months, six months, seven months, eight months, nine months, ten months, eleven months, a year, or chronically. In some embodiments, a chemosensory dysfunction can be ageusia, hypogeusia, dysgeusia, parosmia, phantosmia, anosmia, hyposmia, dysosmia, or any combination thereof. For example, in some embodiments, a chemosensory dysfunction can be anosmia, hyposmia, dysosmia, parosmia, or a combination thereof. In some embodiments, the chemosensory dysfunction can arise after a viral infection. For example, the viral infection can include a coronavirus infection, for example, a SARS-CoV-2 infection or an influenza infection.

In some embodiments, a chemosensory dysfunction can be smell loss, taste loss, or a combination thereof. In some embodiments, a subject before treatment has a decreased level of a cyclic nucleotide or sonic hedgehog or both in a nasal mucus sample from the subject in comparison to a cyclic nucleotide level or a sonic hedgehog level of a control population with normal chemosensory function.

In some embodiments, a method of the disclosure can include administering a second therapeutic. In some embodiments, the second therapeutic can be administered concurrently or consecutively, optionally, wherein the second therapeutic is administered concurrently and is comprised in the gel composition.

In some embodiments, a method of the disclosure can include diagnosing a subject with chemosensory dysfunction. In other embodiments, a subject can be previously diagnosed with chemosensory dysfunction.

In another aspect, this disclosure provides a gel composition useful for treating or preventing chemosensory dysfunction. A gel composition can comprise: (a) a poloxamer; (b) a sucrose acetate isobutyrate (SAIB); or (c) both (a) and (b), and wherein the gel comprises a PDE inhibitor or a salt thereof and wherein the gel composition is configured to release the PDE inhibitor or the salt thereof for over 24 hours.

In a different aspect, this disclosure provides a method of making a gel composition comprising mixing in a mixer: (a) a poloxamer, (b) a sucrose acetate isobutyrate (SAIB), or (c) both (a) and (b), and can include a PDE inhibitor or a salt thereof together to form a gel wherein the gel composition is configured to release the PDE inhibitor or the salt thereof for over 24 hours.

In another aspect, this disclosure provides a kit comprising a gel composition and a container. The gel composition can comprise: (a) a poloxamer; (b) a sucrose acetate isobutyrate (SAIB); or (c) both (a) and (b), and wherein the gel comprises a PDE inhibitor or a salt thereof and wherein the gel composition is configured to release the PDE inhibitor or the salt thereof for over 24 hours.

In another aspect, this disclosure provides a method for delivering the gel composition to a nasal cavity or a sinus cavity of a subject with the use of an applicator or a cannula. The gel composition can comprise: (a) a poloxamer; (b) a sucrose acetate isobutyrate (SAIB); or (c) both (a) and (b), and wherein the gel comprises a PDE inhibitor or a salt thereof and wherein the gel composition is configured to release the PDE inhibitor or the salt thereof for over 24 hours.

Also disclosed herein are methods of treating or preventing chemosensory dysfunction in a subject the method comprising: delivering a gel composition to a nasal cavity or a sinus cavity of the subject to treat or prevent chemosensory dysfunction in the subject. In some embodiments, the gel composition can comprise: a hydroxypropyl cellulose; a polycarbophil; or both. In some embodiments, the gel can comprise a PDE inhibitor or a salt thereof. In some embodiments, the gel is configured to release the PDE inhibitor or the salt thereof for at least about 4 hours. In some embodiments, the gel composition can comprise the hydroxypropyl cellulose and the polycarbophil. In some embodiments, the gel composition can comprise the polycarbophil. In some embodiments, the PDE inhibitor or the salt thereof can comprise theophylline or a salt thereof. In some embodiments, the gel composition can comprise the hydroxypropyl cellulose, the polycarbophil or both independently in an amount from about 0.10% to about 10% weight to weight (wt/wt) of the total gel composition. In some embodiments, the gel composition can comprise the PDE inhibitor or salt thereof in an amount from about 0.01% to about 5% weight to weight (wt/wt) of the total gel composition. In some embodiments, the gel composition can be delivered to the sinus cavity. In some embodiments, the sinus cavity comprises an ethmoid sinus cavity, a maxillary sinus cavity, a frontal sinus cavity, or a sphenoid sinus cavity. In some embodiments, the pharmaceutical can be delivered to the nasal cavity. In some embodiments, the nasal cavity can comprise an upper nasal cavity, an olfactory cleft, an olfactory epithelium or any combination thereof. In some embodiments, the gel composition can be configured to release the PDE inhibitor or the salt thereof over a period of time from about 4 hours to about 14 days. In some embodiments, the gel composition can be in unit dose form. In some embodiments, the gel composition can be administered as needed, or for a time period of about: a week, two weeks, three weeks, a month, two months, three months, four months, five months, six months, seven months, eight months, nine months, ten months, eleven months, a year, or chronically. In some embodiments, the chemosensory dysfunction can be ageusia, hypogeusia, dysgeusia, parosmia, phantosmia, anosmia, hyposmia, dysosmia, or any combination thereof. In some embodiments, the chemosensory dysfunction can be anosmia, hyposmia, dysosmia, parosmia, or a combination thereof. In some embodiments, the chemosensory dysfunction can arise after a viral infection. In some embodiments, the viral infection can comprise a coronavirus infection or an influenza infection. In some embodiments, the chemosensory dysfunction can be smell loss, taste loss, or a combination thereof. In some embodiments, the subject can have a decreased level of a cyclic nucleotide or a sonic hedgehog or both in a nasal mucus sample from the subject in comparison to a cyclic nucleotide level or a sonic hedgehog level of a control population with normal chemosensory function. In some embodiments, the method can further comprise administering a second therapeutic. In some embodiments, the second therapeutic can be administered concurrently or consecutively. In some embodiments, the second therapeutic can be administered concurrently and can be comprised in the gel composition. In some embodiments, the method can further comprise diagnosing the subject with chemosensory dysfunction. In some embodiments, the subject was previously diagnosed with chemosensory dysfunction. In some embodiments, the gel composition can further comprise a phenylethyl alcohol, a citric acid, a sodium chloride, a sodium hydroxide, a water, or any combination thereof.

Also disclosed herein are gel compositions comprising: a hydroxypropyl cellulose; a polycarbophil; or both. In some embodiments, the gel composition can comprise a PDE inhibitor or a salt thereof and the gel composition can be configured to release the PDE inhibitor or the salt thereof for at least about 4 hours. Also disclosed herein are kits and containers comprising the gel compositions disclosed above.

Also disclosed herein are methods making gel compositions comprising mixing in a container: a hydroxypropyl cellulose; a polycarbophil; or both and adding a PDE inhibitor or a salt thereof together to form a gel. In some embodiments, the gel composition is configured to release the PDE inhibitor or the salt thereof for at least about 4 hours.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As recited by in disclosure, various embodiments may be presented in a range format. It should be understood that the description in range format is merely for convenience and brevity and should not be construed as an inflexible limitation on the scope of the disclosure. Accordingly, the description of a range can be considered to have specifically disclosed all the possible subranges as well as individual numerical values within that range. For example, description of a range such as from 1 to 6 can be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This can apply regardless of the breadth of the range.

The singular forms “a”, “an”, and “the” are used herein to include plural references unless the context clearly dictates otherwise. Accordingly, unless the contrary is indicated, the numerical parameters set forth in this application are approximations that can vary depending upon the desired properties sought to be obtained.

The terms “determining”, “measuring”, “evaluating”, “assessing,” “assaying,” and “analyzing” are often used interchangeably herein to refer to forms of measurement and can include determining if an element may be present or not (for example, detection). These terms can include quantitative, qualitative or quantitative, and qualitative determinations. Assessing can be alternatively relative or absolute. “Detecting the presence of” includes determining the amount of something present, as well as determining whether it may be present or absent.

The term “substantially” or “essentially” can refer to a qualitative condition that exhibits an entire or nearly total range or degree of a feature or characteristic of interest. In some cases, substantially can refer to at least about: 70%, 75%, 80%, 85%, 90%, 95%, 99%, or 100% of the total range or degree of a feature or characteristic of interest.

The term “at least partially” can refer to a qualitative condition that exhibits a partial range or degree of a feature or characteristic of interest. In some cases, at least partially can refer to at least about: 5%, 10%, 20%, 30%, 40%, 50%, 60%, 70%, 75%, 80%, 85%, 90%, 95%, 99%, or 100% of the total range or degree of a feature or characteristic of interest.

Unless otherwise indicated, open terms for example “contain,” “containing,” “include,” “including,” and the like can mean comprising.

As used herein, the term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges. The term “substantially” as used herein can refer to a value approaching 100% of a given value. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

When used herein, a percentage of a compound of a composition can be with respect to a total weight or a total volume of a composition. In some cases, a percentage of a component of a composition can be with respect to a total weight or a total volume of a composition

The terms “administer,” “administering”, “administration,” and the like, as used herein, can refer to methods that can be used to enable delivery of compounds or their salts or compositions to the desired site of biological action. Delivery can include direct application to the affect tissue or region of the body. A composition provided herein can be administered by any method. A method of administration can be by inhalation, intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, or any combination thereof. Delivery can include parenteral administration (including intravenous, subcutaneous, intrathecal, intraperitoneal, intramuscular, intravascular or infusion), oral administration, nasal administration, inhalation administration, intraduodenal administration, rectal administration. Delivery can include topical administration (such as a lotion, a cream, a gel, a liquid, a solid, a powder, an ointment) to an external surface of a surface, such as a skin. In some cases, delivery can comprise delivery by a gel, such as an extended release gel. In some case, a gel can be delivered and deposited into a nasal cavity, a sinus cavity or both for a period of time. In some cases, delivery can comprise the use of a cannula or applicator to deliver the gel to a nasal cavity, a sinus cavity or both. In some cases, delivery can comprise the use of an endoscope, microscope and/or image guidance system for delivery to an appropriate anatomic location. In some instances, a subject can administer the gel comprising the compound in the absence of supervision. In some instances, a subject can administer the gel under the supervision of a medical professional (e.g., a physician, nurse, physician's assistant, orderly, hospice worker, etc.). In some cases, a medical professional can administer the gel. In some cases, a cosmetic professional can administer the composition.

As used herein, “treating” of chemosensory dysfunction can include one or more of: reducing the frequency or severity of one or more symptoms, elimination of one or more symptoms or their underlying cause, or improvement or remediation of damage. For example, treatment of chemosensory dysfunction can include, for example, increasing smell acuity and taste acuity from a patient suffering from a viral infection, such as a patient with COVID-19 or influenza and/or causing the regression or disappearance of chemosensory dysfunction.

A “therapeutically effective amount” can refer to an amount of a compound or its salt with or without additional agents that is effective to achieve its intended purpose. Individual patient needs may vary. Generally, the dosage required to provide an effective amount of the compound, salt thereof, or composition containing one or both of these, and which can be adjusted by one of ordinary skill in the art, will vary, depending on the age, health, physical condition, sex, weight, extent of the dysfunction of the recipient, frequency of treatment and the nature and scope of the dysfunction.

The term “subject,” “host,” “individual,” and “patient” are as used interchangeably herein can refer to animals, typically mammalian animals. Any suitable mammal can be administered a compound, salt, or a composition as described herein or treated by a method as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). Mammals can be any age or at any stage of development, for example a mammal can be neonatal, infant, adolescent, adult or in utero. In some embodiments a mammal is a human. Humans can be more than about: 1, 2, 5, 10, 20, 30, 40, 50, 60, 65, 70, 75, 80, 85, 90, 95, 100, 105, 110, 115 or about 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, a human can be less than about 18 years of age. In some cases, human can be from about 1 month to about 12 months old, from about 1 year to about 20 years, from about 15 years to about 50 years, from about 40 years to about 80 years, or from about 60 years to about 110 years. In some cases, a human can be more than about 18 years of age. A mammal such as a human can be male or female. In some embodiments, a subject can have or can be suspected of having a disease or condition. The subject can be a patient, such as a patient being treated for a condition or a disease, such as a heart disease, hypertension, atrial fibrillation, stroke, renal failure, liver disease, cancer, diabetes, respiratory disease, asthma, chronic obstructive pulmonary disease, bronchitis, emphysema, lung cancer, cystic fibrosis, Coronavirus infection, for example, a SARS-CoV-2 infection, a viral infection, a bacterial infection, a fungal infection, a parasitic infection, an influenza infection, pneumonia, pleural effusion, or any combination thereof. A subject can be predisposed to a risk of developing a condition or a disease such as a respiratory disease. A subject can be in remission from a condition or a disease, such as a cancer patient. In some instances, a subject can be healthy.

As disclosed herein, the term “phosphodiesterase (PDE) inhibitor” can refer to a compound that can at least partially inhibit the function of a phosphodiesterase (PDE) polypeptide, such as a PDE1, PDE2, PDE3, PDE4, PDE5 polypeptide, or any combination thereof.

As used herein, reference to a PDE inhibitor generally, or a specific PDE inhibitor, can include any salt, solvate, ester, or polymorph of the PDE inhibitor. A “salt” can include a pharmaceutically acceptable salt. Examples of pharmaceutically acceptable salts can include those salts prepared by reaction of a compound disclosed herein with a mineral, organic acid or inorganic base, such salts including, acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate. Further, a compound disclosed herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. Other acids, such as oxalic, while not in themselves pharmaceutically acceptable, can be employed in the preparation of salts useful as intermediates in obtaining a compound and/or a pharmaceutically acceptable acid addition salt. In some embodiments, a compound disclosed herein which can comprise a free acid group reacts with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts can include the lithium, sodium, potassium, calcium, magnesium, and aluminum salts and the like. Illustrative examples of bases can include sodium hydroxide, potassium hydroxide, choline hydroxide, sodium carbonate, N+(C1-4 alkyl)4, and the like. Representative organic amines useful for the formation of base addition salts can include ethylamine, diethylamine, ethylenediamine, ethanolamine, diethanolamine, piperazine and the like. It may be understood that a compound disclosed herein can also include the quaternization of any basic nitrogen-containing groups they contain. In some embodiments, water or oil-soluble or dispersible products can be obtained by such quaternization. A compound disclosed herein can be prepared as pharmaceutically acceptable salts formed when an acidic proton present in the parent compound either can be replaced by a metal ion, for example an alkali metal ion, an alkaline earth ion, or an aluminum ion; or coordinates with an organic base. In some embodiments, base addition salts can be also prepared by reacting the free acid form of a compound disclosed herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

“Magnitude estimation” or “ME” as used herein can refer to a measurement of the ability of a subject to determine the strength of a stimulant such as an odorant or a tastant.

“Recognition threshold” or “RT” as used herein can refer to a measurement of the ability of a subject to recognize the identity of a stimulant, such as an odorant or a tastant.

“Detection threshold” or “DT” as used herein can refer to a measurement of the ability of a subject to recognize exposure to a stimulant, such as an odorant or a tastant, as being pleasant or unpleasant.

“Hedonic” value or “H” value as used herein can refer to a measurement of a subject's reaction to a stimulant, such as an odorant or a tastant, as being pleasant or unpleasant.

The section headings used herein are for organizational purposes only and are not to be construed as limiting the subject matter described.

### Overview

Disclosed herein are methods of treatment by administering to a subject a phosphodiesterase (PDE) inhibitor, its salt, or a combination of PDE inhibitors or their salts. In some cases, a PDE inhibitor or a salt thereof can be administered as a gel. In some cases, a PDE inhibitor can inhibit a PDE1, a PDE2, a PDE3, a PDE4, a PDE5 polypeptide, or a combination thereof.

PDE2 polypeptides can decrease aldosterone secretion. Such decrease may play an important role in the regulation of elevated intracellular concentrations of cAMP and cGMP in platelets. Several regions of the brain can express PDE2 and rat experiments indicate that inhibition of PDE2 can enhance memory. PDE2 may play a role in regulation of fluid and cell extravasation during inflammatory conditions as PDE2 can be localized to microvessels, especially venous capillary and endothelial cells. PDE2 may also be a good pharmacological target for pathological states such as sepsis or in more localized inflammatory responses such as thrombin-induced edema formation in the lung.

The PDE3 family hydrolyzes cAMP and cGMP, but in a manner suggesting that in vivo, the hydrolysis of cAMP can be inhibited by cGMP. They can also be distinguished by their ability to be activated by several phosphorylation pathways including the PKA and PI3K/PKB pathways. PDE3A can be relatively highly expressed in platelets, as well as in cardiac myocytes and oocytes. PDE3B can be a major PDE in adipose tissue, liver, and pancreas, as well as in several cardiovascular tissues. Both PDE3A and PDE3B can be highly expressed in vascular smooth muscle cells and are likely to modulate contraction. PDE5 can be a regulator of vascular smooth muscle contraction best known as the molecular target for several well-advertised drugs used to treat erectile dysfunction and pulmonary hypertension. In the lung, inhibition of PDE5 can oppose smooth muscle vasoconstriction, and PDE5 inhibitors are in clinical trials for treatment of pulmonary hypertension.

Examples of a PDE inhibitor can include, for example, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI-930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, aminone, tolafentrine, dipyridamole, papaverine, E4021, thienopyrimidine derivatives, triflusal, ICOS-351, tetrahydropiperazino[1,2-b]beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, and the like. In some embodiments, a one or more PDE inhibitors, or their salts, can be formulated in a gel. In some embodiments, the gel can comprise a non-specific PDE inhibitor or its salt. In some embodiments, the gel can comprise an PDE inhibitor or its salt that is selective for a PDE subtype, for example, PDE: 1, 2, 3, 4, or 5. In some embodiments, the gel does not comprise a PDE5 selective inhibitor. In some embodiments, the PDE inhibitor or its salt can be dosed at a range from about 0.001 mg to about: 1 mg, 2 mg, 3 mg, 4 mg, 5 mg, 6 mg, 7 mg, 8 mg, 9 mg, or 10 mg. In some embodiments, the dose can be a total dose released over a 24-hour period of time. For example, a dose of a PED inhibitor or a salt thereof can be about: 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, or 10 mg released in a 24-hour period. In some embodiments, a formulation can be in unit dose form. In some embodiments, a formulation can contain a second active ingredient, such as a corticosteroid, or an antihistamine, or a vasoconstrictor, or any combination thereof. In some embodiments, a formulation does not contain a second active ingredient. In some cases, a formulation can be a pharmaceutical composition such as a gel. In some cases, a composition can be a pharmaceutical composition.

A PDE inhibitor can be a selective PDE inhibitor, or a non-specific PDE inhibitor. A PDE selective inhibitor can include a PDE1 selective inhibitor, PDE2 selective inhibitor, PDE3 selective inhibitor, PDE4 selective inhibitor, or PDE5 selective inhibitor. In some cases, a selective PDE inhibitor can be specific for more than one of PDE1, PDE2, PDE3, PDE4, and PDE5. A non-specific PDE can include a PDE inhibitor that inhibits at least two, three, four, or five of PDE1, PDE2, PDE3, PDE4, and PDE5.

A PDE inhibitor can inhibit cellular apoptosis by inhibiting TNF alpha, TRAIL and their metabolites. PDE inhibitors can activate the production and secretion of nitric oxide in all tissues thereby inducing vasorelaxation or vasodilation of all blood vessels including those of the peripheral blood vessels (inhibiting intermittent claudication), the distal extremities and in the penile region contributing to penile erection.

A non-specific PDE inhibitor can include theophylline, papaverine caffeine, IBMX (3-isobutyl-1-methylxanthine, aminophylline, doxophylline, cipamphylline, theobromine, pentoxifylline (oxpentifylline) and diprophylline. Theophylline can comprise a methylxanthine derivative that, when administered as described herein, can have anti-inflammatory effects on the airways that can be useful to combat the abnormal inflammation seen in asthmatics. In some cases, an anti-inflammatory effect can be achieved when theophylline is prescribed at or administered at levels that produce systemic levels of theophylline in the blood well below that which causes side effects. Patients with emphysema and chronic bronchitis can also be helped with theophylline when their symptoms are partially related to reversible airway narrowing.

A PDE1 selective inhibitor, formerly known as calcium- and calmodulin-dependent phosphodiesterases, can include eburnamenine-14-carboxylic acid ethyl ester (vinpocetine). In some cases, vinpocetine can be used to induce vasorelaxtion on cerebral smooth muscle tissue.

A PDE2 selective inhibitor can include EHNA (erythro-9-(2-hydroxy-3-nonyl) adenine), 9-(6-phenyl-2-oxohex-3-yl)-2-(3,4-dimethoxybenzyl)-purin-6-one (PDP), and BAY 60-7750.

A PDE3 selective inhibitor can include enoximone, milrinone (Primacor), amrinone, cilostamide, cilostazol (Pletal) and trequinsin. A PDE3 inhibitor when administered as described herein can produce sympathetic stimulation to increase cardiac inotropy, chronotropy and dromotropy. A PDE3 inhibitor when administered as described herein can also antagonize platelet aggregation, increase myocardial contractility, and enhance vascular and airway smooth muscle relaxation. PDE3A can be a regulator of this process. A PDE3 inhibitor when administered as described herein can effectively prevent aggregation. Cilastazol (Pletal), is approved for treatment of intermittent claudication. Its mechanism of action may involve inhibition of platelet aggregation along with inhibition of smooth muscle proliferation and vasodilation.

A PDE4 selective inhibitor can include mesembrine, rolipram, Ibudilast (i.e. a neuroprotective and bronchodilator drug that can be used in the treatment of asthma and stroke), and roflumilast (Daxas) and cilomilast (Airflo) (i.e. PDE4 selective inhibitors that can be administered for treatment of chronic obstructive pulmonary disease). A PDE4 selective inhibitor can at least partially suppress release of inflammatory mediators e.g., cytokines, or at least partially inhibit production of reactive oxygen species and immune cell infiltration. A PDE4 inhibitor can also be used to treat asthma, arthritis, and psoriasis.

A PDE5 selective inhibitor can include Sildenafil, tadalafil, vardenafil, udenafil and avanafil.

As described herein a gel can comprise a PDE inhibitor or a salt thereof. In some cases, a gel can be a semisolid system comprising a dispersion made up of particles enclosing or interpenetrated by a liquid. Gels can be a cross-linked system that exhibits little to no flow when in the steady-state. In some cases, a gel can comprise a continuous structure that provides solid-like properties. For example, a gel can be a natural or synthetic polymer that forms a three-dimensional matrix though a dispersion medium in which a drug can be dispersed. In some cases, a PDE inhibitor or salt thereof can also be formulated for topical delivery as a cream or ointment. The gels described herein can be administered topically for a localized or system effect. For example, a gel comprising a PDE inhibitor such as theophylline or a salt thereof can be administered to a nasal or a sinus cavity to provide localized treatment for chemosensory dysfunction. In some cases, a gel can be applied to an absorbable or non-absorbable packing material. In some cases, a gel can be applied to a delivery tool (e.g., an applicator) or to a reservoir. In some cases, a gel can be placed adjacent to the site of delivery. In some cases, a gel can be placed directly at the site of delivery. In some instances, topical administration can bypass first pass metabolism. In some cases, a gel described herein can be a single-phase gel, or a two-phase gel. A single-phase gel may not have discrete particles while a two-phase gel may have discrete particles. In some cases, a gel described herein can comprise a gel forming agent, a penetration enhancer, and an active ingredient. In some cases, a gel can further comprise a filler such as water or other organic compositions. In some cases, a gel can be made by mixing in a mixer or a blender a gel forming agent (e.g., a poloxamer and/or a sucrose acetate isobutyrate), an active ingredient (e.g., a PDE inhibitor or a salt thereof), a penetration enhancer or a combination of any of these. In some embodiments, a gel can be formed in vivo or ex vivo. For example, a gel can be formed by mixing two or more compositions in vivo for increased stability. In some cases, a gel can be formed by mixing two or more compositions up to 24 hours prior to administration for increased stability and/or shelf life.

In some embodiments, a gel described herein can comprise a luteolin, a salt thereof, or a derivative thereof. In some cases, a gel described herein can comprise a palmitoylethanolamide, a derivative thereof, or a salt thereof. In some cases, a gel described herein can comprise a palmitoylethanolamide, a derivative thereof, or a salt thereof and a luteolin, a derivative thereof, or a salt thereof. In some cases, a palmitoylethanolamide, a derivative thereof, or a salt thereof and/or a luteolin a derivative thereof, or a salt thereof can comprise an active agent in a gel. In some cases, a palmitoylethanolamide, a derivative thereof, a salt thereof, a luteolin, a derivative thereof, or a salt thereof can be mixed with a PDE inhibitor or salt thereof. In some cases, the PDE inhibitor can comprise theophylline or a salt thereof. In some cases, a palmitoylethanolamide, a derivative thereof, or a salt thereof, and a luteolin, a derivative thereof, or a salt thereof can be mixed with a PDE inhibitor or salt thereof. In some cases, a palmitoylethanolamide, a derivative thereof, or a salt thereof and/or a luteolin a derivative thereof, or a salt thereof can be used to treat a chemosensory dysfunction. In some instances, a palmitoylethanolamide, a derivative thereof, a salt thereof, a luteolin, a derivative thereof, a salt thereof, a PDE inhibitor or a salt thereof, can each independently be present in a therapeutically effective amount to treat a chemosensory dysfunction. In some cases, a PDE inhibitor or a salt thereof can be administered intranasally and a palmitoylethanolamide, a derivative thereof, a salt thereof, a luteolin, a derivative thereof, a salt thereof, or any combination thereof can be administered orally, for example in the form of a pill, a liquid, a capsule or a tablet. In some embodiments representative dosages of a palmitoylethanolamide, a derivative thereof, a salt thereof, a luteolin, a derivative thereof, or a salt thereof can independently be from about 1.0 μg to 2000 mg per day, from about 1.0 μg to 500.0 mg per day, from about 10 μg to 100.0 mg per day, from about 10 μg to about 10 mg per day, from about 10 μg to 1.0 mg per day, from about 10 μg to 500 μg per day, from about 20 μg to about 2000 μg per day, from about 100 μg to about 10,000 μg per day, or from about 1 μg to 50 μg per day. These ranges of dosage amounts represent total dosage amounts of the active ingredient per day for a given patient. In some embodiments, a daily administered dose can be less than about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, a daily administered dose can be at least about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day.

In some embodiments, a gel described herein can comprise a PDE inhibitor or a salt thereof, such as theophylline or a salt thereof. In some cases, a gel can comprise a gel forming agent (e.g., a polymer), a pharmaceutical drug, a penetration enhancer or any combination thereof. In some cases, a gel formulation will not react physically, chemically or both with a drug formulated within the gel. In some cases, an excipient may be added to a gel. In some cases, a co-solvent may be added to the gel for example, ethanol, propylene glycol, or PEG. In some cases, a gel can comprise an inorganic gel or an organic gel. In some cases, a gel can be a hydrogel or an organogel.

In some cases, a gel can comprise water in an amount from about 1% to about 99% weight to weight (wt/wt) of the total composition. In some cases, a gel can comprise water in an amount of about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 3²%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% (wt/wt) of the total composition.

In some embodiments a gel can comprise a gel forming agent. A gel forming agent can be an agent that is added to a gel formulation to help the gel assemble a cohesive internal structure. In some embodiments, a gel forming agent can comprise a natural polymer, a synthetic polymer, an inorganic substance, or a combination thereof. In some cases, a natural polymer can comprise a protein, a collagen, a gelatin, a xanthin, a gellum gum, a natural gum, or any combination thereof. In some cases, a natural polymer can comprise a polysaccharide. In some cases, a gel can comprise a sucrose acetate isobutyrate (SAIB). In some instances, a polysaccharide can comprise an agar, a alginic acid, a sodium carrageenan, a carrageenan, a potassium carrageenan, a tragacanth, a pectin, a guar gum, a cassia tora, or a combination thereof. In some cases, a gel forming agent can comprise a cellulose derivative, for example a carboxymethyl cellulose, a methylcellulose, a hydroxypropyl cellulose, a hydroxypropyl methylcellulose, a hydroxyethyl cellulose or a combination thereof. In some cases, a gel forming agent can comprise a synthetic polymer. In some instances, a synthetic polymer can comprise a carbomer (e.g., carbopol-910, carbopol-940, carbopol-934, carbopol-934P, carbopol-941), a poloxamer, a polyacrylamide, a polyvinyl alcohol, a polyethylene, a copolymer of polyethylene or any combination thereof. In some cases, a gel forming agent can comprise an inorganic substance, for example aluminum hydroxide, an aluminum salt, bentonite, veegum, laponite, or a combination thereof. In some instances, a gel forming agent can comprise a cetosteryl alcohol, a brij-96. In some cases, a gel forming agent can comprise a carbomer (e.g., a carbopol). For example a carbomer can comprise any one of the following carbopol polymers: 71G NF, 971P NF, 974P NF, 980 NF, 981 NF, 5984 EP, ETD 2020 NF, Ultrez 10 NF, 934 NF, 934P NF, 940 NF, 941 NF, 1342 NF, or any combination thereof. In some instances, a gelling agent can comprise a poloxamer, such as P105, P108, P122, P123, P124, P182, P183, P184, P185, P188, P212, P217, P234, P235, P237, P238, P288, P333, P334, P335, P338, P402, P403, P407 or any combination thereof. In some cases, a gel forming agent can comprise hydroxypropyl cellulose (HPC). In some cases, HPC can comprise HPC-H. In some cases, HPC can comprise HPC-EL, HPC-E, HPC-L, HPC-J, HPC-G, HPC-M, HPC-R, HPC-H, or any combination thereof. In some instances, a gel can comprise a peptide. In some cases, a collagen can comprise atelocollagen. In some instances, a gel can have porous structure such as a microporous or nanoporous structure. In some cases, a cream or an ointment can comprise a gel forming agent. In some cases, a gel can comprise a gel thickener. In some cases, a gel thickener can comprise a hydroxypropyl cellulose. In some cases, a gel forming agent can comprise a gel thickener. In some cases, a gel forming agent can be used interchangeably with a gel thickener.

In some embodiments, a gel can comprise an environmental responsive gelling agent. For example, an environmental responsive gelling agent can have controlled gel forming characteristics in response to an environmental stimulus, such as a temperature change. In some cases, a gel can be a thermo-sensitive gel, a light-sensitive gel, a pH sensitive gel, an ultrasound sensitive gel or any combination thereof.

In some embodiments, a gel can comprise a gel forming agent in an amount from about 0.01% to about 30% weight to weight (wt/wt) of the total composition. In some cases, a gel can comprise a gel forming agent in an amount from about: 0.01% to about 1%, 1% to about 10%, 2% to about 8%, 5% to about 15%, 4% to about 12%, 1% to about 20%, 10% to about 20%, or 15% to about 30% (wt/wt) of the total composition. In some cases, a gel can comprise a gel forming agent in an amount of about: 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% (wt/wt) of the total composition. In some cases, a gel can comprise one gel forming agent. In some cases, a gel can comprise more than one gel forming agent, for example a gel can comprise 2, 3, 4, 5, or more gel forming agents.

In some embodiments, a gel described herein can comprise a penetration enhancer. In some cases, a penetration enhancer can be a substance that is capable or promoting penetration of a drug into the skin, mucous membrane, nerve sheath or through another barrier (e.g., a mucosal tissue). In some cases, a penetration enhancer can be at least partially: inert, non-toxic, non-irritating, non-allergenic, compatible with a drug and excipients, odorless, tasteless, colorless or any combination thereof. In some cases, a penetration enhancer can be a fatty acid, an alcohol, a surfactant, a solvent, a hydrogen bond acceptor or any combination thereof. In some instances, a penetration enhancer can comprise an azonem (1-dodecylazacycloheptan-2-one), dimethylsulfoxide, dimethylacetamide, dimethylformamide, ethanol, propylene glycol, N-methyl pyrrolidone, oleic acid, a lauryl alcohol, a ketone terpene, a terpene, a sulfoxide, an alkanol, an organic acid, an alcohol, a polyol, pyrrolidone, a glycol, urea and derivatives of urea, an enzyme, a iminosulfurane, a cyclodextrin, a fatty acid ester, a surfactant, a polymer, a monoolein, a oxalidinone, or any combination thereof. In some cases, a cream or an ointment can comprise a penetration enhancer.

In some embodiments, a gel described herein can comprise a mucosal adhesive. In some cases, a mucosal adhesive can comprise a penetration enhancer. In some cases, a penetration enhancer can comprise a mucosal adhesive. In some cases, a mucosal adhesive can comprise Noveon AA1. In some cases, a mucosal adhesive can comprise a polycarbophil or a derivative thereof. In some cases, a mucosal adhesive can comprise a carbomer homopolymer or a derivative thereof. In some cases, a carbomer homopolymer can comprise carbomer homopolymer Type A, carbomer homopolymer Type B, or a combination of both.

In some embodiments, a gel can comprise a penetration enhancer in an amount from about 1% to about 20% weight to weight (wt/wt) of the total composition. In some cases, a gel can comprise a penetration enhancer in an amount from about: 0.1% to 10%, 10% to about 10%, 2% to about 8%, 5% to about 15%, 4% to about 12% or 10% to about 20% (wt/wt) of the total composition. In some cases, a gel can comprise a penetration enhancer in an amount of about: 0.1%, 0.2%, 0.30%, 0.4%, 0.50%, 0.6%, 0.7%, 0.80%, 0.9%, 1%, 2%, 3%, 4%, 5%, 6%, 7%, 8%, 9%, 10%, 11%, 12%, 13%, 14%, 15%, 16%, 17%, 18%, 19%, or 20% (wt/wt) of the total composition. In some cases, a gel can comprise one penetration enhancer. In some cases, a gel can comprise more than one penetration enhancer, for example a gel can comprise 2, 3, 4, 5, or more different penetration enhancers.

In some cases, a hydrogel can comprise ingredients that are at least partially soluble in water. In some cases, a hydrogel can comprise hydrophilic colloids such as silica, bentonite, tragacanth, pectin, sodium alginate, methylcellulose carboxymethylcellulose sodium, alumina, or any combination thereof. In some instances, a gel can comprise methylcellulose, hydroxy ethylcellulose, and sodium carboxymethyl cellulose (CMC). In some cases, an organogel can comprise hydrocarbons, fats (e.g., animal and vegetable fats), lipids, or a combination thereof.

In some cases, a gel can comprise an active pharmaceutical ingredient such as a PDE inhibitor or salt thereof (e.g., theophylline, a salt thereof, cilostazol, a salt thereof, or a combination thereof). In some cases, a gel can comprise an active pharmaceutical ingredient in an amount from about 0.001% to about 20% weight to weight (wt/wt) of the total composition. In some cases, a gel can comprise an active pharmaceutical ingredient in an amount from about: 0.001% to about 0.01%, 0.01% to about 0.1%, 0.01% to about 1%, 0.1% to about 1%, 1% to about 10%, 2% to about 8%, 5% to about 15%, 4% to about 12% or 10% to about 20% (wt/wt) of the total composition. In some cases, a gel can comprise an active pharmaceutical ingredient in an amount of about: 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20% (wt/wt) of the total composition. In some cases, a gel can comprise one an active pharmaceutical ingredient. In some cases, a gel can comprise more than one active pharmaceutical ingredient, for example a gel can comprise 2, 3, 4, 5, or more different an active pharmaceutical ingredients.

In some cases, a gel described herein can comprise an odor or a scent. For example, an odor or a scent can comprise a floral scent, a perfume, a fruity scent, a citrus scent (e.g., lemon, lime, orange), a wood scent, woody (e.g. pine or fresh cut grass), a chemical scent (e.g., ammonia, bleach), a sweet scent (e.g., chocolate, vanilla, caramel), a minty scent (e.g., and peppermint, eucalyptus and camphor), a toasted and nutty scent (e.g., popcorn, peanut butter, almonds) or a pungent scent (e.g., blue cheese, tobacco). In some cases, a gel can comprise a tastant. In some cases, a tastant can comprise a sweet tastant (e.g., a sugar), salty (e.g., a salt), umami, bitter, or sour. In some cases, a cream or an ointment can comprise a scent or tastant.

In some embodiments, a gel described herein can comprise a preservative. In some cases, a preservative can comprise phenylethyl alcohol (PEA). In some embodiments, a preservative can be methylparaben, propylparaben, a quaternary ammonium compound, benzalkonium chloride or any combination thereof. In some cases, a pH of a formulation can be maintained from about 4.5 to about 7.0, or from about 5.0 to about 7.0 or from about 5.5 to about 6.5. In some cases, the osmolarity of a formulation can also be adjusted to osmolarities of from about 250 to about 350 mOsm/L. In some cases, a gel can comprise a preservative, a buffering agent, a tonicity agent, and/or a pH agent in an amount of about: 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, (wt/wt) of the total composition. In some cases, a gel can comprise a preservative, a buffering agent, a tonicity agent, and/or a pH agent in an amount of about: 0.001% to about 0.01%, 0.01% to about 0.1%, 0.01% to about 1%, 0.1% to about 1%, 1% to about 10%, or 2% to about 8% (wt/wt) of the total composition.

In some cases, a gel can comprise theophylline as an active agent, a phenylethyl alcohol as a preservative, a citric acid as a buffering agent, a sodium chloride as a tonicity agent, a sodium hydroxide as a pH agent, a polycarbophil as a mucosal adhesive, a hydroxypropyl cellulose as a gel thickener, and water as a solvent.

In some cases, a gel disclosed herein can have a viscosity level. In some cases, the viscosity level of a gel can be about: 100 centipoise (cP), 200 cP, 300 cP, 400 cP, 500 cP, 600 cP, 700 cP, 800 cP, 900 cP, 1000 cP, 2000 cP, 3000 cP, 4000 cP, 5000 cP, 6000 cP, 7000 cP, 8000 cP, 9000 cP, 10,000 cP, 15,000 cP, 20,000 cP, 25,000 cP, 30,000 cP, 35,000 cP, 40,000 cP, 45,000 cP, 50,000 cP, 55,000 cP, 60,000 cP, 65,000 cP, 70,000 cP, 75,000 cP, 80,000 cP, 85,000 cP, 90,000 cP, 95,000 cP, 100,000 cP, 250,000 cP, 500,000 cP, or greater than 500,000 cP. In some cases, a gel disclosed herein can have a viscosity level of about: 50 cP to about 1,000,000 cP, 50 cP to about 1,000 cP, 100 cP to about 500 cP, 250 cP to about 3000 cP, 500 cP to about 5000 cP, 1000 cP to about 6000 cP, 2500 cP to about 10,000 cP, 10,000 cP to about 30,000 cP, 50,000 cP to about 1000,000 cP, 50,000 cP to about 250,000 cP, 100,000 cP to about 500,000 cP, or 500,000 cP to about 1,000,000 cP.

In some cases, a gel can be a delivery system configured to release a drug for a specific time period. For example, a hydrogel can be configured to release theophylline or a salt thereof for an extended period of time. In some cases, a gel can be configured to release a drug (such as a PDE inhibitor) for about: 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, or more than 6 months. In some cases, a gel can be configured to release a drug (such as a PDE inhibitor) for at least about: 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, or 6 months. In some cases, a gel can be configured to release a drug (such as a PDE inhibitor) for about: 1 hour to about 24 hours, 1 hour to about 5 hours, 2 hours to about 8 hours, 3 hours to about 6 hours, 4 hours to about 8 hours, 4 hours to about 15 hours, 5 hours to about 18 hours, 7 hours to about 20 hours, 10 hours to about 24 hours, 10 hours to about 15 hours, 24 hours to about 48 hours, 1 day to about 4 days, 2 days to about 6 days, 3 days to about 6 days, 1 day to about 7 days, 1 day to about 14 days, 7 days to about 14 days, 1 week to about 3 weeks, 2 weeks to about 5 weeks, 3 weeks to about 6 weeks or 1 month to about 6 months. In some cases, a drug will have at least partially first order release kinetics or zero order release kinetics, or a combination thereof. For example, on initial application a drug applied in gel form can have first order release kinetics followed by zero order release kinetics. In some cases, a gel can be configured to release an active agent with substantially zero order kinetics. In some cases, a gel can be configured to release an active agent with substantially first order kinetics.

When administered in vivo, a gel described herein can be administered in combination with one or more pharmaceutically acceptable carriers or excipients and in dosages described herein. A gel can be formulated as pharmaceutically acceptable a neutral (free base) or a salt form. In some embodiments, a pharmaceutically acceptable carrier can include but are not limited to: an amino acid, a peptide, a protein, a non-biological polymer, a biological polymer, a simple sugar, a carbohydrate, a gum, an inorganic salt and a metal compound which may be present singularly or in combination. In some embodiments, a pharmaceutically acceptable carrier can comprise native, derivatized, a modified form, or combinations thereof.

In some embodiments, a composition or formulation can include an excipient. Excipients can include, but are not limited to one or more of: water, a fluidizer, a lubricant, an adhesion agent, a surfactant, an acidifying agent, an alkalizing agent, an agent to adjust pH, an antimicrobial preservative, an antioxidant, an anti-static agent, a buffering agent, a chelating agent, a humectant, a gel-forming agent, or a wetting agent. Excipients can also include a coloring agent, a coating agent, a sweetening agent, a flavoring and perfuming agent or a masking agent. A composition and formulation can include a therapeutic agent with an individual excipient or with multiple excipients in any suitable combination, with or without a carrier. In some cases, an excipient can comprise glycerol. In some cases, a gel described herein can comprise a buffering agent. In some cases, a buffering agent can comprise citric acid. In some cases, a buffering agent can comprise a phosphoric acid, an acetic acid, a histidine lactic acid, a tromethamine gluconic acid, an aspartic acid, a glutamic acid, a tartaric acid, a succinic acid, a malic acid, a fumaric acid, an alpha ketoglutaric acid or any combination thereof. In some cases, a gel described herein can comprise a tonicity agent. In some cases, a tonicity agent can comprise sodium chloride. In some cases, a tonicity agent can comprise a dextrose, a glycerin, a mannitol, a potassium chloride, or any combination thereof. In some cases, a gel described herein can comprise a pH agent. In some cases, a pH agent can comprise sodium hydroxide (NaOH). In some cases, a pH agent can comprise hydrochloric acid (HCl).

In some cases, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

Also disclosed herein are methods of making compositions disclosed herein. In some cases, a method can comprise mixing components of a composition, such as those found in Table 2 and/or those described herein, in a mixer or container. In some cases, a method of making can comprise contacting components of a mixture to form a gel. In some cases, a gel can be packaged in a container.

### Methods of Treatment

Disclosed herein are methods for treating chemosensory dysfunction in a human. In some cases, the chemosensory dysfunction can be at least in part produced from, or occurring during or after, a viral infection (e.g., a coronavirus infection, an influenza infection). In some cases, chemosensory dysfunction can be at least in part produced from an allergy, an inflammation, a traumatic accident, a neurodegenerative disorder, or an idiopathic cause. In some cases, the chemosensory dysfunction can be at least in part produced from, or occurring during or after, damage to the nervous system (e.g., the sensory nervous system). In some cases, the chemosensory dysfunction can be at least in part produced from, or occurring during or after, a coronavirus infection or a mutated form thereof. In some cases, a coronavirus can comprise SARS-CoV-2 or a mutated form thereof, which can cause the disease COVID-19. In some cases, the method can comprise administering to a subject a therapeutically effective amount of a phosphodiesterase (PDE) inhibitor or a pharmaceutically acceptable salt thereof to treat chemosensory dysfunction. In some cases, a PDE inhibitor can be administered in the form of a gel, for example a gel formulated for extended release. In some cases, a PDE inhibitor or pharmaceutically acceptable salt thereof can be administered in a formulation in unit dose form.

In some cases, a disease or condition described herein can be chemosensory dysfunction. In some cases, chemosensory dysfunction can comprise at least partial: loss of the sense of taste, loss of the sense of smell or both. Chemosensory dysfunction can comprise a taste or smell disorder. In some cases, a taste or smell disorder can comprise anosmia, hyposmia, ageusia, hypogeusia, dysosmia, phantosmia, dysgeusia, parosmia, or a combination thereof.

The methods of treatment can include by way of example only, oral administration, transmucosal administration, buccal administration, nasal administration such as inhalation, parental administration, intravenous, subcutaneous, intramuscular, sublingual, transdermal administration, and rectal administration. In some cases, administration can comprise administration to a nasal cavity or a sinus cavity. In some cases, administration can comprise administration the ear, the eyes, the mouth or a combination thereof. In some instances, a sinus cavity can comprise an ethmoid sinus cavity, a maxillary sinus cavity, a frontal sinus cavity, or a sphenoid sinus cavity. In some instances, administration into a sinus cavity can comprise administration to a sinus ostium. The nasal cavity is a space that extends from the nares to the nasopharynx. The medial boundary is the nasal septum, the inferior boundary is the nasal floor. The lateral boundary includes the lateral nasal wall including the turbinates. The spaces between the turbinates and the lateral nasal wall including the inferior meatus, middle meatus, superior meatus and sphenoethmoid recess are within the nasal cavity. The superior boundary is defined by the skull base formed from the frontal bone, the cribiform plate of the ethmoid and the sphenoid bone. The olfactory nerves can be found in the superior aspect of the nasal cavity within the olfactory region/olfactory cleft below the cribiform plate. In some embodiments, a nasal cavity can comprise an upper nasal cavity, an olfactory cleft, an olfactory epithelium or any combination thereof. In some cases, the nasal cavity can comprise the squamous mucosa, the olfactory mucosa, the respiratory mucosa, or a combination thereof. In some cases, the nasal cavity can comprise the superior turbinate, the middle turbinate, the inferior turbinate or a combination thereof. In some embodiments, the composition can be administered as a liquid nasal wash, an aerosol, a powder aerosol or a combination thereof. In some cases, the administering can comprise application intranasally in one nostril or both nostrils. In some embodiments, a gel composition can be administered to the naris, a nasal cavity, a sinus cavity or a combination thereof. In some cases, a gel can be administered to a naris, a nasal cavity, a sinus cavity or a combination thereof. In some cases, a gel can be administered to substantially all of a nasal cavity or a sinus cavity. In some cases, a gel can be administered to at least part of a nasal cavity or a sinus cavity. In some cases, a gel can be administered to the ear, an eye, the mouth or a combination thereof. In some embodiments, the liquid composition can be administered as a nasal wash. In some cases, administering a gel can be performed during, after or both during and after a viral infection.

In some embodiments, a PDE inhibitor or a salt thereof can be directly applied to the nasal or lingual epithelium as a liquid, cream, lotion, ointment, or a gel. These can contain at least one therapeutically active PDE inhibitor or its salt or any active agent. In some cases, the formulations can further include at least one excipient that can be formulated for administration. In some cases, a gel can be applied with an applicator or without an applicator to at least a portion of one or more: nasal cavities, sinus cavities or any combination thereof. In some cases, an applicator can be a device used to apply a gel, for example a cotton swab or a cannula. In some cases, an application device can comprise a swab. In some cases, delivery can comprise the use of an endoscope, microscope and/or image guidance system for delivery to an appropriate anatomic location. In some cases, an applicator can apply a gel described herein to a nasal cavity or a sinus cavity.

In some embodiments, compositions such as gels, lotions, or ointments described herein when stored in a sealed container and placed in a room at 25° C. and a room atmosphere having about 50 percent relative humidity, can retain at least about: 80%, 90%, 95%, 96%, 97%, 98%, or 99% of the active ingredient or the salt thereof after 6 months, as measured by HPLC.

In some embodiments, the subject can be administered an olfactory assay that measures: threshold, discrimination, identification or any combination thereof. In some instances, an olfactory assay can be administered prior to, during, or after treatment. In some cases, a threshold assay can be used to determine the lowest concentration of an odorant that can be reliably detected. In some cases, a discrimination assay can be used to assess the ability of a subject to distinguish two or more different smells. In some cases, an identification assay can be used to assess the ability of a subject to identify a specific odor. In some cases, an olfactory test can be used to determine the efficacy of a treatment such as treatment with a gel comprising a PDE inhibitor. For example, an olfactory assay can be completed prior to treatment and after treatment to determine a measurable change in a subject's chemosensory dysfunction.

In some embodiments, a method of treatment can comprise olfactory training (e.g., smell training). In some cases, olfactory training, which can involve repetitive stimulation of peripheral olfactory neurons, may rely on the regenerative capacity of superior olfactory pathways. In some cases, olfactory training can comprise exposure to 1, 2, 3, 4, 5, 6 or more than 6 different odors. In some cases, olfactory training can comprise exposure to the different odors 1, 2, 3, 4 or more than 4 times per day. In some cases, olfactory training can take place for about: 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, or longer than 1 year. In some cases, olfactory training can take place for about: 7 days to about 21 days, 21 days to about 120 days, 30 days to about 90 days, 1 month to about 5 months, 2 months to about 3 months, 3 months to about 8 months, 6 months to about 1 year, 8 months to about 18 months, or 1 year to about 3 years. In some cases, organic essences of lemon, rose, eucalyptus and cloves can be used as the different odors for olfactory testing.

In some embodiments, a subject can experience a change (e.g., a decrease) in: a detection threshold (DT) score, a recognition threshold (RT) score, or both. In some embodiments, a subject can experience a change (e.g., an increase) in a magnitude estimation (ME) score. In some cases, a subject can experience a change in a hedonic (H) score. In some cases, the changes in RT score, ME score, DT score, or H score can be measured with a forced-choice, three-stimuli, stepwise-staircase technique using one or more odorants after the administration of a PDE inhibitor to a subject. In some cases, the one or more odorants comprise pyridine, nitrobenzene, thiophene, amyl acetate, or a combination thereof.

In some embodiments, the subject can experience a change (e.g., a decrease) in: a taste detection threshold (DT) score, a recognition threshold (RT) score, or both. In some embodiments, a subject can experience a change (e.g., an increase) in a magnitude estimation (ME) score. In some cases, a subject can experience a change in a hedonic (H) score. In some cases, the changes in RT score, ME score, DT score, or H score can be measured with a forced-choice, three-stimuli, stepwise-staircase technique using one or more tastants testing compounds after administration of a PDE inhibitor to the subject. In some cases, the one or more tastants comprise sodium chloride (NaCl), sucrose, hydrogen chloride (HCl), urea, or a combination thereof.

In some embodiments, a subject can experience a clinically detectable improvement in taste or smell function within about: 1 week to about 6 weeks, 1 week to about 4 weeks, 2 weeks to about 5 weeks, or about 3 weeks to about 4 weeks of starting treatment. In some embodiments, a subject can experience a clinically detectable improvement in taste or smell function within about: 1 month to about 6 months, 1 month to about 4 months, 2 months to about 5 months, or about 3 months to about 4 months of starting treatment.

In some cases, administering a PDE inhibitor as described herein can be used to prevent or treat diseases or conditions associated with or caused by a viral infection, a bacterial infection, a fungal infection, a parasitic infection or any combination thereof. In some cases, administering a PDE inhibitor as described herein can be used to prevent or treat diseases or conditions associated with the nervous system, such as the sensory nervous system, the central nervous system, or the peripheral nervous system. In some cases, a viral infection can comprise coronavirus. In some cases, a viral infection can comprise an influenza virus. Such diseases and conditions can include, for example, anosmia, taste loss, smell loss, hyposmia, ageusia, dysosmia, parosmia, phantosmia, chemosensory dysfunction, cough, fever, fever, malaise, difficult breathing, runny nose, sore throat, nasal congestion or any combination thereof. In some cases, an influenza infection can be caused by Influenza A, Influenza B (e.g., B (Victoria), B (Yamagata)). In some cases, Influenza A can comprise Influenza H1N1, H3N2, a mutation of any of these, or any combination thereof. In some cases, an influenza virus can comprise one or more mutations. In some cases, a coronavirus infection can be caused by alpha coronavirus, beta coronavirus, gamma coronavirus, delta coronavirus, 229E coronavirus, NL63 coronavirus, OC43 coronavirus, HKU1 coronavirus, MERS-CoV, SARS-CoV, SARS-CoV-2, a mutated form thereof, or any combination of these. In some cases, a coronavirus can cause COVID-19. In one example, a gel described herein can be used to treat chemosensory dysfunction from SARS-CoV-2 or a mutated form thereof. In some cases, a virus, such as a coronavirus or an influenza virus can have a mutation. For example, a coronavirus or an influenza virus can comprise about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotide mutations as compared to a reference sequence. In some cases, a coronavirus or an influenza virus can comprise a genome with more than about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity or sequence similarity as compared to a reference genome sequence. In some cases, a coronavirus or an influenza virus can comprise a genome with less than about: 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity or sequence similarity as compared to a reference genome sequence. In some cases, a reference sequence can be a reference sequence from the National Center for Biotechnology Information.

In some cases, methods or compositions of the disclosure as described herein can be used to treat a subject inflicted with a chemosensory disorder. A chemosensory disorder can include a loss of smell (anosmia), or a reduced ability to smell (hyposmia). A chemosensory disorder can include a loss of taste (ageusia), or a reduced ability to taste (hypogeusia), for example, a reduced ability to taste a sweet, sour, bitter, or salty substance. In some instances, a chemosensory disorder involves odors or tastes or flavors to be misread or distorted. For example, a chemosensory disorder can cause a person to detect an unpleasant odor or taste from something that is normally pleasant to taste or smell.

A chemosensory disorder (e.g., can accompany, or be associated with,) a disease or condition, such as, obesity, diabetes, hypertension, malnutrition, or a degenerative disease of the nervous system, such as, Parkinson's disease, Alzheimer's disease, or multiple sclerosis. In some embodiments, a chemosensory disorder can be associated with an allergy such as allergic rhinitis. In some instances, an allergy can comprise a drug allergy, a food allergy, an insect allergy, a latex allergy a mold allergy, a pet allergy, a pollen allergy, or a combination thereof. In some embodiments, a chemosensory disorder can be associated with an inflammation. In some cases, inflammation can comprise sinusitis, mucosal inflammation, rhinosinusitis, nasal polyposis or any combination thereof. In some embodiments, a chemosensory disorder can be associated with a trauma. In some cases, trauma can comprise a traumatic brain injury (TBI), a head injury, a concussion, or any combination thereof.

Disclosed herein are methods of treating a condition (e.g., taste and smell loss) by administering a PDE inhibitor or salt thereof as described herein. In some cases, administering can comprise administering a PDE inhibitor or salt thereof in unit dose form, for example in a gel. In some embodiments representative daily intranasal, lingual, pulmonary, topical or mucosal dosages are from about 1.0 μg to 2000 mg per day, from about 1.0 μg to 500.0 mg per day, from about 10 μg to 100.0 mg per day, from about 10 μg to about 10 mg per day, from about 10 μg to 1.0 mg per day, from about 10 μg to 500 μg per day, from about 20 μg to about 2000 μg per day, from about 100 μg to about 10,000 μg per day, or from about 1 μg to 50 μg per day of the active ingredient comprising a compound (i.e. PDE inhibitor). These ranges of dosage amounts represent total dosage amounts of the active ingredient per day for a given patient. In some embodiments, a daily administered dose can be less than about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, a daily administered dose can be at least about: 2000 mg per day, 1000 mg per day, 500 mg per day, 100 mg per day, 10 mg per day, 1.0 mg per day, 500 μg per day, 300 μg per day, 200 μg per day, 100 μg per day or 50 μg per day. In some embodiments, on a per kilo basis, suitable dosage levels of a compound can be from about 0.001 μg/kg to about 10.0 mg/kg of body weight per day, from about 0.5 μg/kg to about 0.5 mg/kg of body weight per day, from about 1.0 μg/kg to about 100 μg/kg of body weight per day, and from about 2.0 μg/kg to about 50 μg/kg of body weight per day. In some embodiments, a suitable dosage level on a per kilo basis can be less than about: 10.0 mg/kg of body weight per day, 1 mg/kg of body weight per day, 500 μg/kg of body weight per day, 100 μg/kg of body weight per day, 10 μg/kg of body weight per day, or 1.0 μg/kg of body weight per day of the compound. In some embodiments, a suitable dosage level on a per kilo basis can be at least about: 10.0 mg/kg of body weight per day, 1 mg/kg of body weight per day, 500 μg/kg of body weight per day, 100 μg/kg of body weight per day, 10 μg/kg of body weight per day of the active ingredient, or 1.0 μg/kg of body weight per day of the compound.

In some cases, the amount administered can be the same amount administered to treat a particular disease or can be an amount lower than the amount administered to treat that particular disease. The dosage may be administered once per day or several or multiple times per day. For example, a gel can be administered once per day or once every 2 days. In another example, a PDE inhibitor or salt thereof can be administered 2, 3, 4, 5, 6, 7, 8, 9, 10 or more times per day. In some cases, a composition can be administered once, twice or thrice in a 24-hour period. In some cases, a gel can be configured to release a PDE inhibitor or salt thereof for a period of time. In some cases, a gel may be administered daily or less frequently in a sustained release formulation. For example, a gel can be administered every: 1 day, 2 days, 3 days, 4 days, 5 days, 6 days, 7 days, 1 week, 2 weeks, 3 weeks, 4 weeks, 5 weeks, 1 month, 2 months, 2 months, 3 months, 4 months, 5 months, 6 months, 7 months, 8 months, 9 months, 10 months, 11 months, 12 months, or more than 12 months. In some cases, a composition can be administered 1, 2, 3, 4, 5 or more than 5 times in a: 2 day, 3 day, 4 day, 5 day, 6 day, week, 2 week, 3 week, or 4 week period of time. In some cases, a gel can be administered as needed. The amount of the drug administered to practice methods of the present disclosure will of course, be dependent on the subject being treated, the severity of the affliction, the manner of administration and the judgment of the prescribing physician. The dose used to practice the disclosure can produce the desired therapeutic or prophylactic effects, without producing serious side effects.

In some embodiments, administration of an effective amount of a PDE inhibitor by intranasal, lingual, pulmonary, topical, or mucosal administration does not produce a detectable blood level of the PDE inhibitor. In some embodiments, administration of an effective amount of a PDE inhibitor by intranasal, lingual, pulmonary, topical, or mucosal administration produces blood concentration of the PDE inhibitor that are less than about: 5 mg/dl, 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl. In some embodiments, administration of an effective amount of a PDE inhibitor by intranasal, lingual, pulmonary, topical, or mucosal administration produces blood concentration of the PDE inhibitor that are more than about: 2 mg/dl, 1 mg/dl, 500 μg/dl, 250 μg/dl, 100 μg/dl, 50 μg/dl, 25 μg/dl, 10 μg/dl, 5 μg/dl, or 1 μg/dl.

In some embodiments, administration of an effective amount of a PDE inhibitor or salt thereof can increase the salivary and/or nasal mucus cAMP or cGMP levels in the human by at least about: 5%, 6%, 7%, 8%, 9%, 10, 1%, 1²%, 13%, 14%, 1%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of PDE inhibitor or pharmaceutically acceptable salt thereof. In some cases, the increase of the salivary and/or nasal mucus cAMP or cGMP levels are observed after about: 1 to about 10 days, 30 to about 90 days, 15 to about 45 days, or 30 days of continuous treatment with the therapeutically effective amount of PDE inhibitor or pharmaceutically acceptable salt thereof.

In some embodiments, administration of an effective amount of a PDE inhibitor or a salt thereof in the form of a gel can increase taste or smell acuity. In some embodiments, an increase in taste or smell acuity can be at least about: 5%, 10%, 20%, 30%, 40%, 50%, 75%, or 100% compared to the untreated state. In some embodiments, taste or smell acuity can be increased to at least about: 5%, 10%, 15%, 20%, 25%, 30%, 35%, 40%, 45%, 50%, 55%, 60%, 65%, 70%, 75%, 80%, 85%, 90%, 95% or 100% of the acuity of normal individuals. In some cases, an increase in taste or smell acuity can be measured after about: 10 to about 20 days, 15 to about 30 days, 25 days to about 50 days, 1 month to about 6 months, 4 months to 12 months, 6 months to 18 months, or 6 months to about 3 years. In some cases, an increase in taste or smell acuity can be measured after about 30 days. In some embodiments, taste or smell acuity can be measured objectively. In some embodiments, taste or smell acuity can be measured subjectively. In some cases, smell acuity can be measured by detection threshold, recognition threshold, hedonics, magnitude estimation or any combination thereof.

In some cases, a composition described herein can be administered with one or more additional therapeutics. For example, a PDE inhibitor or salt thereof can be administered with a second therapy. In some cases, a second therapy can be administered concurrently or consecutively. In some cases, the second therapeutic can be administered concurrently and is comprised in the gel composition. In some cases, the second therapeutic can be administered concurrently and is not comprised in the gel composition. In some cases, an additional therapeutic can comprise an antihistamine, diphenhydramine, chlorpheniramine, cetirizine, desloratadine, fexofenadine, levocetirizine, loratadine, azelastine, olopatadine, ketotifen, olopatadine, pheniramine, a decongestant, pseudoephedrine, oxymetazoline, tetrahydrozoline, a corticosteroid, budesonide, fluticasone furoate, fluticasone propionate, mometasone, triamcinolone, beclomethasone, ciclesonide, budesonide, fluticasone, mometasone, fluorometholone, loteprednol, prednisone, betamethasone, desonide, hydrocortisone, mometasone, triamcinolone, cromolyn, lodoxamide, nedocromil, an immunotherapy, a salt of any of these, or any combination thereof. In some cases, a second therapy can comprise remdesivir, a salt thereof, chloroquine, a salt thereof, lopinavir, a salt thereof, ritonavir, a salt thereof, favilavir, a salt thereof, interferon beta, a salt thereof, an antiviral, oxygen or any combination thereof. In some cases, an additional therapeutic can comprise nitric oxide, a steroid, a non-steroidal anti-inflammatory drug (NSAID), or any combination thereof. In some cases, an additional therapy can be comprised in a gel that comprises a PDE inhibitor.

In some cases, a subject can be diagnosed (e.g. diagnosed with chemosensory dysfunction) prior to treatment with a PDE inhibitor or salt thereof. In some cases, a method of treatment can comprise diagnosing chemosensory dysfunction in a subject. In some cases, the diagnosing can comprise an in vitro assay. In some cases, chemosensory dysfunction can be diagnosed by detecting sonic hedgehog at or below a threshold level in a biological sample from the human. In some cases, chemosensory dysfunction can be diagnosed by cyclic AMP (cAMP), cyclic GMP (cGMP), or both at or below a threshold level in a biological sample from the human. In some cases, a sample can be a nasal sample or a saliva sample. In some cases, chemosensory dysfunction can be diagnosed by detecting a cyclic nucleotide level at or below a threshold level in the biological sample from the human. In some cases, chemosensory dysfunction can be diagnosed by determining a detection threshold, by determining a recognition threshold, and by magnitude estimation for at least one of: pyridine, nitrobenzene, thiophene, and amyl acetate. In some cases, diagnosis can comprise detecting a RT score, a ME score, a DT score, a H score or a combination thereof and comparing to a reference population (e.g., a population without chemosensory dysfunction).

The Hedgehog signaling pathway is commonly referenced as a key regulator of animal development, particularly during late stages of embryogenesis and metamorphosis. Mammals may have three Members of the hedgehog signaling pathway, Sonic Hedgehog (SHH), Desert Hedgehog (DHH), and Indian hedgehog (IHH). The pathway has been implicated in the development of some cancers. Members of the hedgehog signaling pathway can be used in diagnosing and treating loss and/or distortion of taste or smell, e.g., hyposmia, dysosmia, anosmia, phantosmia, hypogeusia, dysgeusia, phantogeusia, and/or ageusia.

The term “one or more members of the hedgehog signaling pathway” as used herein and its grammatical equivalents can include known or unknown members of the hedgehog signaling pathway. For example, known members of the hedgehog signaling pathway can include the currently known members of the hedgehog signaling pathway, Sonic Hedgehog (SHH), Desert Hedgehog (DHH), and Indian hedgehog (IHH). Unknown members of the hedgehog signaling pathway can be found by comparing the homology of nucleic acid and proteins sequences. Although, the disclosure may be directed towards any member of the hedgehog signaling pathway, specific hedgehog members can be of significant influence. Therefore, it is contemplated that the disclosure can focus on SHH, DHH, IHH, or any combination thereof. For example, the embodiments disclosed herein can be focused on SHH.

The one or more members of the hedgehog signaling pathway can be selected from a group consisting of: Sonic Hedgehog (SHH), Desert Hedgehog (DHH), and/or Indian hedgehog (IHH). The one or more members of the hedgehog signaling pathway can be SHH, DHH, IHH, or any combination thereof. Although a mammalian (e.g., human) hedgehog can be measured, it is also contemplated that a non-mammalian hedgehog can be measured. In some cases, chemosensory dysfunction in a subject can be determined by detecting a level of Sonic Hedgehog (SHH) that ranges from about greater than 0 μg/mL to about 8,500 μg/mL; a level of Indian hedgehog (IHH) that ranges from about greater than 0 μg/mL about to 1.0 μg/mL; or a level of Desert Hedgehog (DHH) that ranges from about greater than 0 μg/mL to about 5.0 μg/mL, or a combination thereof.

The levels of members of the hedgehog signaling pathway in patients exhibiting loss and/or distortion of taste or smell (e.g., hyposmia, dysosmia, anosmia, phantosmia, hypogeusia, dysgeusia, phantogeusia, and/or ageusia) can be lower than normal controls. For example, in patients suffering from loss and/or distortion of taste or smell (e.g., chemosensory dysfunction), the level of SHH, in some cases, can be or about: 0 μg/mL, greater than 0 μg/mL to less than less than 1 μg/mL, 1 μg/mL to 25 μg/mL, 15 μg/mL to 30 μg/mL, 20 μg/mL to 40 μg/mL; 35 μg/mL to 50 μg/mL; 45 μg/mL to 100 μg/mL; 75 μg/mL to 150 μg/mL, 125 μg/mL to 1000 μg/mL, 900 μg/mL to 2500 μg/mL, 2000 μg/mL to 5000 μg/mL, 4000 μg/mL to 7500 μg/mL, 6000 μg/mL to 10,000 μg/mL; (b) the level of IHH can be or about: 0 μg/mL, greater than 0 μg/mL to 0.1 μg/mL, 0.05 μg/mL to 0.15 μg/mL, 0.125 μg/mL to 0.2 μg/mL, 0.15 μg/mL to 0.30 μg/mL, 0.25 μg/mL to 0.5 μg/mL, 0.4 μg/mL to 0.7 μg/mL, 0.6 μg/mL to 0.75 μg/mL, 0.725 μg/mL to 0.9 μg/mL, 0.8 μg/mL to 1.0 μg/mL, less than 1.0 μg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL; (c) the level of DHH can be or about: 0 μg/mL, greater than 0 μg/mL to 0.1 μg/mL, 0.05 μg/mL to 0.15 μg/mL, 0.125 μg/mL to 0.2 μg/mL, 0.15 μg/mL to 0.30 μg/mL, 0.25 μg/mL to 0.5 μg/mL, 0.4 μg/mL to 0.7 μg/mL, 0.6 μg/mL to 0.75 μg/mL, 0.725 μg/mL to 0.9 μg/mL, 0.8 μg/mL to 1.0 μg/mL, 0.9 μg/mL to 1.1 μg/mL, 1.0 μg/mL to 1.3 μg/mL, 1.2 μg/mL to 1.5 μg/mL, 1.4 μg/mL to 2.0 μg/mL, 1.9 μg/mL to 2.5 μg/mL, 2.4 μg/mL to 3.0 μg/mL, 2.9 μg/mL to 3.5 μg/mL, 3.4 μg/mL to 3.8 μg/mL, 3.7 μg/mL to 3.9 μg/mL, 3.85 μg/mL to 5.0 μg/mL, less than 5.0 μg/mL, less than 0.05 ng/mL, less than 0.15 ng/mL, less than 0.2 ng/mL, less than 0.3 ng/mL, less than 0.5 ng/mL, less than 0.7 ng/mL, less than 0.75 ng/mL, less than 0.9 ng/mL, less than 1.0 ng/mL, less than 1.1 ng/mL, less than 1.5 ng/mL, less than 1.75 ng/mL, less than 2.0 ng/mL, less than 2.25 ng/mL, less than 5.0 ng/mL, less than 6.0 ng/mL, less than 7.0 ng/mL, less than 10.0 ng/mL, or less than 100.0 ng/mL. However, there can be some inter-patient variability because the levels of the different members of the hedgehog signaling pathway vary based on the person.

In some embodiments, administration of an effective amount of a PDE inhibitor can increase the salivary and/or nasal mucus SHH, DHH, and/or IHH levels in the human by at least about: 5%, 6%, 7%, 8%, 9%, 10%, 11%, 12%, 13%, 14%, 15, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of PDE inhibitor. In some cases, the increase of the salivary and/or nasal mucus SHH, DHH, and/or IHH levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor can increase or decrease a biological compound, such as a protein or a metabolite. In some embodiments, administration of an effective amount of a PDE inhibitor can increase the salivary and/or nasal mucus cAMP or cGMP levels in the human by at least about: 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or about 50% relative to these levels in the human before the administering of the therapeutically effective amount of the PDE inhibitor. In some cases, the increase of the salivary and/or nasal mucus cAMP or cGMP levels are observed after about: 1 to about 10 days, 30 to about 90 days, 15 to about 45 days, or 30 days of continuous treatment with the therapeutically effective amount of a PDE inhibitor.

In some embodiments, administration of an effective amount of a PDE inhibitor can decrease the salivary and/or nasal mucus IL-10 levels in the human by at least about: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 41%, 42%, 43%, 44%, 45%, 46%, 47%, 48%, 49%, or about 50% relative to these levels in the human before the administering of the therapeutically effective amount of the PDE inhibitor. In some cases, the decrease of the salivary and/or nasal mucus IL-10 levels are observed after about: 1 to about 10 days, 30 to about 90 days, 15 to about 45 days, or 30 days of continuous treatment with a therapeutically effective amount of a PDE inhibitor. IL-10 levels can be measured with an enzyme-linked immunoassay (ELISA), Western blot, or other protein measurement assay.

To assess if a subject has loss and/or distortion of taste or smell, e.g., hyposmia, dysosmia, anosmia, phantosmia, hypogeusia, dysgeusia, phantogeusia, and/or ageusia, based on levels of one or more members of the hedgehog signaling pathway, a threshold comparison, e.g., a basal level can be used. Thus, the threshold level can be an average level for one or more members of the hedgehog signaling pathway as measured in a control population comprising subjects with normal olfactory and/or gustatory function. The level of one or more members of the hedgehog signaling pathway can be at least one order of magnitude lower than said threshold level. For example, 2, 3, 4, 5, 10, 15, 16, 17, 18, 19, 20, 21, 22, 23, 24, 25, 26, 26, 27, 28, 29, 30, 31, 32, 33, 34, 35, 36, 37, 38, 39, 40, 41, 42, 43, 44, 45, 50, 60, 70 80, 90, 100, 150, 200, 250, 300, 400, 500, 600, 700, 800, 900, or 1000 or more orders of magnitude lower than said threshold level.

Also described herein are kits comprising a gel, a lotion or an ointment. In some cases, a kit can comprise a container that comprises a gel, a lotion or an ointment. In some instances, a kit can comprise instructions for use. In some instances, a container can be a sterile container. In some cases, a container can be a plastic, a glass, or a metal container. In some instances, a kit can comprise an applicator and/or cannula for delivery.

According to some aspects of the disclosure, provided herein is a pharmaceutical gel composition useful to treat or prevent chemosensory dysfunction of a subject. In some instances, the pharmaceutical gel pharmaceutical composition can include a PDE inhibitor or the salt thereof and can be configured to release the PDE inhibitor or the salt thereof for at least 24 hours. In some instances, the gel composition can be formulated to achieve a zero-order release. Accordingly, in some instances the pharmaceutical gel composition can be configured to release the PDE inhibitor or the salt thereof at a constant rate for a period of time of, for example, up to 2 hours, 4 hours, 6 hours, 10 hours, 12 hours, or 24 hours. In some instances, the zero-order release pharmaceutical gel composition can be formulated with a methacrylic acid copolymer either alone or in combination with a cellulose polymer. In some instances, the pharmaceutical gel composition can be homogenous throughout. In other instances, the pharmaceutical composition can be heterogenous throughout. The pharmaceutical gel composition can be administered to a nasal cavity.

### NUMBERED EMBODIMENTS

A number of compositions, methods, and kits are disclosed herein. Specific exemplary embodiments of these of compositions, methods, and kits are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A method of treating or preventing chemosensory dysfunction in a subject the method comprising: delivering a gel composition to a nasal cavity or a sinus cavity of the subject to treat or prevent chemosensory dysfunction in the subject, wherein the gel composition comprises: a) a hydroxypropyl cellulose; b) a polycarbophil; or c) both (a) and (b), and wherein the gel comprises a PDE inhibitor or a salt thereof and wherein the gel is configured to release the PDE inhibitor or the salt thereof for at least about 4 hours.

Embodiment 2. The method of embodiment 1, comprising the hydroxypropyl cellulose.

Embodiment 3. The method of embodiment 1, wherein the PDE inhibitor or the salt thereof comprises theophylline or a salt thereof.

Embodiment 4. The method of embodiment 1, wherein the gel composition comprises the hydroxypropyl cellulose, the polycarbophil, or both, independently in an amount from about 0.1% to about 10% weight to weight (wt/wt) of the total gel composition.

Embodiment 5. The method of embodiment 1, wherein the gel composition comprises the PDE inhibitor or salt thereof in an amount from about 0.01% to about 5% weight to weight (wt/wt) of the total gel composition.

Embodiment 6 The method of embodiment 1, wherein the gel composition is delivered to the sinus cavity and wherein the sinus cavity comprises an ethmoid sinus cavity, a maxillary sinus cavity, a frontal sinus cavity, or a sphenoid sinus cavity.

Embodiment 7. The method of embodiment 1, wherein the gel composition is delivered to the nasal cavity and wherein the nasal cavity comprises an upper nasal cavity, an olfactory cleft, an olfactory epithelium or any combination thereof.

Embodiment 8. The method of any one of embodiments 1-7, wherein the gel composition is configured to release the PDE inhibitor or the salt thereof over a period of time from about 4 hours to about 14 days.

Embodiment 9. The method of any one of embodiments 1-8, wherein the gel composition is in unit dose form.

Embodiment 10. The method of any one of embodiments 1-9, wherein the gel composition is administered as needed, or for a time period of about: a week, two weeks, three weeks, a month, two months, three months, four months, five months, six months, seven months, eight months, nine months, ten months, eleven months, a year, or chronically.

Embodiment 11. The method of any one of embodiments 1-10, wherein the chemosensory dysfunction is ageusia, hypogeusia, dysgeusia, parosmia, phantosmia, anosmia, hyposmia, dysosmia, or any combination thereof.

Embodiment 12. The method of any one of embodiments 1-10, wherein the chemosensory dysfunction is anosmia, hyposmia, dysosmia, parosmia, or a combination thereof.

Embodiment 13. The method of embodiment 1, wherein the chemosensory dysfunction arises after or during a viral infection.

Embodiment 14. The method of embodiment 13, wherein the viral infection comprises a coronavirus infection or an influenza infection.

Embodiment 15. The method of embodiment 1, wherein the chemosensory dysfunction is smell loss, taste loss, or a combination thereof.

Embodiment 16. The method of any one of embodiments 1-15, wherein the subject before treatment has a decreased level of a cyclic nucleotide or a sonic hedgehog or both in a nasal mucus sample from the subject in comparison to a cyclic nucleotide level or a sonic hedgehog level of a control population with normal chemosensory function.

Embodiment 17. The method of any one of embodiments 1-16, further comprising administering a second therapeutic.

Embodiment 18. The method of embodiment 17, wherein the second therapeutic is administered concurrently or consecutively, optionally, wherein the second therapeutic is administered concurrently and is comprised in the gel composition.

Embodiment 19. The method of any one of embodiments 1-18, further comprising diagnosing the subject with chemosensory dysfunction.

Embodiment 20. The method of any one of embodiments 1-18, wherein the subject was previously diagnosed with chemosensory dysfunction.

Embodiment 21. The method of embodiment 1, comprising the gel composition which comprises the polycarbophil.

Embodiment 22. The method of embodiment 1, wherein the gel composition further comprises a phenylethyl alcohol, a citric acid, a sodium chloride, a sodium hydroxide, a water, or any combination thereof.

### EXAMPLES

### Example 1

A patient diagnosed or previously diagnosed with parosmia is treated with an intranasally administered hydrogel comprising theophylline. The hydrogel is administered to the nasal cavity of the patient. The hydrogel comprises a polyethylene glycol (PEG) mixture and theophylline. The hydrogel is configured to release theophylline at a dosage of about 0.25 μg/kg to 2.6 μg/kg per day for about 7 days.

Administration of the hydrogel produces an increase in the patient's recognition threshold and detection threshold within about 30 days. Further, this increase produces a significant increase in the patient's ability to smell, thus at least partially ameliorating the parosmia.

### Example 2

An extended-release gel that delivers a PDE inhibitor is used to treat anosmia in a subject having recovered from COVID-19, where the anosmia was caused COVID-19 and persists despite recovery from COVID-19. The extended-release gel is configured to provide a dosage unit of theophylline, roflumilast, cilostazol, or a combination thereof, when administered to a nasal cavity or a sinus cavity. The dosage unit comprises an effective amount of the theophylline, roflumilast, cilostazol, or a combination thereof in the extended-release gel.

After administration of the dosage unit using extended-release gel, the patient is examined and retreated over a 60-day period to monitor the treatment of the anosmia using an increase in the recognition threshold as compared to prior to the first administration.

### Example 3

A patient diagnosed or previously diagnosed with dysgeusia is treated with an intranasally administered hydrogel comprising theophylline. The gel is administered to the nasal cavity of the patient. The hydrogel comprises a poloxamer mixture and theophylline. The gel is configured to release theophylline at a dosage of about 15 μg to about 150 μg per day for about 4 days.

Administration of the gel produces an increase in the patient's recognition threshold and detection threshold within about 60 days. Further, this increase produces a significant increase in the patient's ability to taste, thus at least partially ameliorating the dysgeusia.

### Example 4

A patient diagnosed or previously diagnosed with a coronavirus infection and having at least partial chemosensory dysfunction associated with the coronavirus infection is treated with an intranasally administered hydrogel comprises a polyethylene glycol (PEG) mixture and theophylline. The hydrogel is configured to release theophylline at a dosage of about 500 μg per day for about 4 days.

Administration of the gel produces an increase in the patient's cAMP levels within about 90 days. Further, this increase produces a significant increase in the patient's ability to taste and smell, thus at least partially ameliorating the chemosensory dysfunction.

### Example 5

The Patient initially reported their sensory dysfunction as either loss of taste (i.e., flavor) and/or smell function. This subjective response was documented by objective psychophysical measurements of olfactory function administered to each patient by use of a forced-choice, three-stimuli, stepwise-staircase technique in a fixed, controlled design (1, 2). Efficacy of this technique and results of testing were previously documented in a double-blind clinical trial (2). Four odors were used; they were pyridine (dead-fish odor), nitrobenzene (bitter-almond odor), thiophene (petroleum-like odor) and amyl acetate (banana-oil odor). Detection thresholds (DT), and recognition thresholds (RT) values for each odor were determined as previously described (1, 2). Thresholds were converted into bottle units (BU) as previously described (2) and results reported as M of correct responses for each odor in each treatment group. References: (1) Henkin, R. I. Evaluation and treatment of human olfactory dysfunction, in Otolaryngology (English, G. M. Ed.), Lip pincott, Philadelphia, 1993, Vol. 2, pp. 1-86. (2) Henkin, R. I., Schecter, P. J., Friedewald, W. T., DeMets, D. L., Raff, M. S. A double blind study of the effects of zinc sulfate on taste and smell dysfunction. Amer. J. Med. Sci. 1976; 272: 285-299.

Theophylline was administered to a patient who had loss of taste and smell from COVID-19. A formulation containing 80 μg of theophylline was administered via two actuations from nasal spray device once per day. Prior to the initial administration of theophylline, a baseline smell test was performed. A follow-up smell test was performed 3 months after the initial administration. No side effects were reported. The results are shown in Table 1. The patient's detection threshold (DT) and recognition threshold (RT) for pyridine (Pyr), nitrobenzene (NO2B), thiophene (Thio) and amyl acetate (AA) was determined before treatment (pre) and after treatment (3M). In all four odorant tests, the patient's detection threshold and recognition threshold improved. An improvement of 1 bottle unit means that a patient could detect and/or recognize an odorant that was 10 times lower in concentration, when compared to baseline. For example, the recognition threshold (RT) of AA improved by 8 bottle units, also equivalent to an improvement of 8 orders of magnitude. In this case, the patient could detect a concentration of AA that was 108 lower in concentration. Bottle unit numbers that are 5 or lower are considered to be in the range of normal.

TABLE 1

| | | DT | DT | DT | DT | RT | RT | RT | RT |
|---|---|---|---|---|---|---|---|---|---|
| | odor | Pyr | NO2B | Thio | AA | Pyr | NO2B | Thio | AA |
| Patient | Pre | 11 | 11 | 10 | 11 | 11 | 12 | 10 | 12 |
| Patient | 3 months | 7 | 9 | 4 | 4 | 7 | 9 | 4 | 4 |

DT = Detection Threshold;
RT = Recognition Threshold

### Example 6

A gel comprising a PDE inhibitor was formulated for intranasal use. The gel formulation is described in Table 2. The gel contained theophylline as the active agent, phenylethyl alcohol as a preservative, citric acid as a buffering agent, sodium chloride as a tonicity agent, sodium hydroxide as a pH agent, noveon AA1 (a polycarbophil) as a mucosal adhesive, hydroxypropyl cellulose (HPC-H as a_gel thickener, and water as a solvent

TABLE 2
Theophylline gel formulation

| Ingredient | Function | grams | % |
|---|---|---|---|
| Theophylline USP | Active ingredient | 0.310 | 0.30% |
| Phenylethyl alcohol | Preservative | 0.200 | 0.20% |
| (PEA) | | | |
| Citric acid | Buffering agent | 0.030 | 0.03% |
| Sodium chloride | tonicity agent | 0.250 | 0.24% |
| NaOH 1% | pH agent | 0.95 | 0.93% |
| Noveon AA1 | Mucosal | 0.51 | 0.50% |
| (polycarbophil) | adhesive | | |
| Hydroxypropyl cellulose | Gel thickener | 1.90 | 1.85% |
| (HPC)-H | | | |
| Water | Solvent | 98.28 | 95.95% |
| Total | | 102.430 | 100.00% |

### Example 7

A gel comprising the PDE inhibitor described in Example 6 and Table 2 was administered to an adult male suffering from chronic smell loss. The gel was administered to the male's nose by a swab to deliver it to the olfactory region. The patient suffered no adverse effects. The patient demonstrated the ability to smell post administration of the gel.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating a chemosensory dysfunction in a subject the method comprising: delivering a gel composition to a nasal cavity or a sinus cavity of the subject to treat the chemosensory dysfunction in the subject, wherein the gel composition comprises:
a) a hydroxypropyl cellulose in an amount from about 0.1% to about 10% weight to weight (wt/wt) of the total gel composition;
b) a polycarbophil in an amount from about 0.1% to about 10% weight to weight (wt/wt) of the total gel composition; or
c) both (a) and (b),
and wherein the gel comprises a PDE inhibitor or a salt thereof and wherein the gel is configured to release the PDE inhibitor or the salt thereof for at least about 4 hours after delivering the gel composition to the nasal cavity or the sinus cavity of the subject.

2. The method of claim 1, wherein the gel composition comprises the hydroxypropyl cellulose and the polycarbophil.

3. The method of claim 1, wherein the PDE inhibitor or the salt thereof comprises theophylline or a salt thereof.

4. The method of claim 1, wherein the gel composition comprises the hydroxypropyl cellulose in an amount of about 1.85% weight to weight (wt/wt) of the total gel composition.

5. The method of claim 1, wherein the gel composition comprises the PDE inhibitor or salt thereof in an amount from about 0.01% to about 5% weight to weight (wt/wt) of the total gel composition.

6. The method of claim 1, wherein the gel composition is delivered to the sinus cavity and wherein the sinus cavity comprises an ethmoid sinus cavity, a maxillary sinus cavity, a frontal sinus cavity, or a sphenoid sinus cavity.

7. The method of claim 1, wherein the gel composition is delivered to the nasal cavity and wherein the nasal cavity comprises an upper nasal cavity, an olfactory cleft, an olfactory epithelium or any combination thereof.

8. The method of claim 1, wherein the gel composition is configured to release the PDE inhibitor or the salt thereof over a period of time from about 4 hours to about 14 days after delivering the gel composition to the nasal cavity or the sinus cavity of the subject.

9. The method of claim 1, wherein the gel composition is in unit dose form.

10. The method of claim 1, wherein the gel composition is administered as needed, or for a time period of about: a week, two weeks, three weeks, a month, two months, three months, four months, five months, six months, seven months, eight months, nine months, ten months, eleven months, a year, or chronically.

11. The method of claim 1, wherein the chemosensory dysfunction is ageusia, hypogeusia, dysgeusia, parosmia, phantosmia, anosmia, hyposmia, dysosmia, or any combination thereof.

12. The method of claim 1, wherein the chemosensory dysfunction is anosmia, hyposmia, dysosmia, parosmia, or any combination thereof.

13. The method of claim 1, wherein the chemosensory dysfunction arises after or during a viral infection, and wherein the viral infection comprises a coronavirus infection or an influenza infection.

14. (canceled)

15. The method of claim 1, wherein the chemosensory dysfunction is smell loss, taste loss, or a combination thereof.

16. (canceled)

17. The method of claim 1, further comprising administering a second therapeutic.

18.-20. (canceled)

21. The method of claim 1, wherein the gel composition comprises the polycarbophil.

22. The method of claim 1, wherein the gel composition further comprises a phenylethyl alcohol, a citric acid, a sodium chloride, a sodium hydroxide, a water, or any combination thereof.

23. A gel composition comprising:
(a) a hydroxypropyl cellulose in an amount from about 0.1% to about 10% weight to weight (wt/wt) of the total gel composition;
(b) a polycarbophil in an amount from about 0.1% to about 10% weight to weight (wt/wt) of the total gel composition; or
(c) both (a) and (b),
and wherein the gel composition comprises a PDE inhibitor or a salt thereof and wherein the gel composition is configured to release the PDE inhibitor or the salt thereof for at least about 4 hours after delivering the gel composition to a nasal cavity or a sinus cavity of a subject.

24. A kit comprising the gel composition of claim 23 and a container.

25. A method of making a gel composition comprising contacting in a container:
(a) a hydroxypropyl cellulose in an amount from about 0.1% to about 10% weight to weight (wt/wt) of the total gel composition;
(b) a polycarbophil in an amount from about 0.1% to about 10% weight to weight (wt/wt) of the total gel composition; or
(c) both (a) and (b),
and a PDE inhibitor or a salt thereof together to form a gel wherein the gel composition is configured to release the PDE inhibitor or the salt thereof for at least about 4 hours after delivering the gel composition to a nasal cavity or a sinus cavity of a subject.

26.-74. (canceled)
